(19) United States
(12) Patent Application Publication
(10) Pub. No.: US 2023/0304991 A1
(43) Pub. Date: Sep. 28, 2023

(54) INTOXICATION DETECTION DEVICE

(71) Applicant: TOYODA GOSEI CO., LTD., Kiyosu-shi (JP)

(72) Inventors: Rie ONO, Aichi-ken (JP); Itsuhiko HIROTA, Aichi-ken (JP); Hiroshi TAKAKURA, Aichi-ken (JP)

(21) Appl. No.: 18/188,590

(22) Filed: Mar. 23, 2023

(30) Foreign Application Priority Data
Mar. 25, 2022 (JP) 2022-050578

(51) Int. Cl. G01N 33/497

(52) U.S. Cl. CPC G01N 33/4972; B62D 1/06

(57) ABSTRACT

An intoxication detection device includes: a gas detection sensor; a gas storage recess which is recessed to accommodate the gas detection sensor therein; and an exhaust fan formed adjacent to the gas storage recess. The intoxication detection device is configured to detect an alcohol component contained in a discharge gas discharged from a skin of the driver without contacting the skin in response to a hand of the driver being brought close to the gas detection sensor. The exhaust fan is operated for a predetermined period of time while a state of the hand blocking the gas storage recess is detected, and during the operation, the exhaust fan is configured to exhaust air or the discharge gas stored in the gas storage recess. The gas detection sensor is set so as to measure the discharge gas stored in the gas storage recess after the exhaust fan stops operating.

### CROSS-REFERENCE TO RELATED APPLICATION(S)

This application is based upon and claims the benefit of priority from prior Japanese patent application No. 2022-050578 filed on Mar. 25, 2022, the entire contents of which are incorporated herein by reference.

### BACKGROUND

### 1. Field of the Invention

The present invention relates to an intoxication detection device which is mounted near a driver's seat in a vehicle and capable of detecting the intoxication of a driver.

### 2. Description of the Related Art

In the related art, an intoxication detection device detects the intoxication of a driver by measuring an atmospheric alcohol concentration in the driver's seat in which the driver is seated, and also measuring a body temperature and a heart rate of the driver (for example, see JP-A-2019-91232). There is also an intoxication detection device that detects the intoxication of a driver by measuring a concentration of alcohol component in the driver's perspiration (see JP-A-2009-73422).

Meanwhile, the intoxication detection device described in JP-A-2019-91232 is not only configured to measure the driver's body temperature and heart rate, but also configured to detect the alcohol component by measuring the atmospheric alcohol concentration in the driver's seat, and accordingly, there is a possibility that the alcohol component not discharged from the driver, such as the alcohol component from alcohol disinfectant sprays and air fresheners, or the alcohol component contained in the exhale of an occupant in the passenger seat can be detected, and there is a concern of misdetecting the intoxication of the driver. Further, the intoxication detection device described in JP-A-2009-73422 detects the intoxication of a driver by measuring a concentration of alcohol component in the perspiration of the driver gripping a steering wheel, in which case the detection device detects the alcohol component discharged from the driver, but according to this configuration, since the driver has to perspire by means of a perspiration booster so that the concentration of the alcohol component in the perspiration is measured, it is not possible to detect the alcohol component quickly.

### SUMMARY

The present invention is provided to resolve the issues described above, and accordingly, it is an object of the present invention to provide an intoxication detection device capable of preventing misdetection and accurately detecting the intoxication of a driver.

According to an aspect of the present disclosure, there is provided an intoxication detection device mounted near a driver's seat in a vehicle and configured to detect intoxication of a driver, the intoxication detection device including: a gas detection sensor; a gas storage recess which is recessed to accommodate the gas detection sensor therein; and an exhaust fan formed adjacent to the gas storage recess, where he intoxication detection device is configured to detect an alcohol component contained in a discharge gas discharged from a skin of the driver without contacting the skin in response to a hand of the driver being brought close to the gas detection sensor, where the gas storage recess is configured to store the discharge gas therein as the hand covers a peripheral edge in contact therewith, where the exhaust fan is operated for a predetermined period of time while a state of the hand blocking the gas storage recess is detected, and during the operation, the exhaust fan is configured to exhaust air or the discharge gas stored in the gas storage recess, and where the gas detection sensor is set so as to measure the discharge gas stored in the gas storage recess after the exhaust fan stops operating.

According to the aspect, the intoxication detection device is configured to store the discharge gas discharged from the hand of the driver in the gas storage recess accommodating the gas detection sensor therein, and detect the alcohol component from the discharge gas, in which the intoxication detection device includes the exhaust fan and is configured to operate the exhaust fan to exhaust air or the discharge gas stored in the gas storage recess while the opening area of the gas storage recess is blocked with the hand, and then measure, by the gas detection sensor, the discharge gas discharged from the hand of the driver and stored in the gas storage recess. Therefore, it is possible to prevent the detection of alcohol components caused by the factors other than the driver, since it is possible to measure the discharge gas discharged from the hand of the driver in a state in which, for example, any air already stored in the gas storage recess before the hand of the driver comes into contact, or any discharge gas remaining in the gas storage recess after a predetermined period of time from the previous measurement is exhausted (removed), such that it is possible to accurately detect the alcohol component discharged from the driver.

Therefore, according to the prevent invention, the intoxication detection device can prevent misdetection and accurately detect the intoxication of the driver.

Further, in the intoxication detection device of the present invention, it is preferable to control operations such that the gas detection sensor measures the alcohol component in the discharge gas a plurality of times within a predetermined period of time, because it is possible to prevent misdetection and detect the intoxication of the driver more accurately and stably.

Furthermore, in the intoxication detection device having the configuration described above, it is preferable that the gas storage recess is disposed in a grip portion gripped by the driver in the steering wheel steered by the driver, because the gas storage recess can be stably blocked with the driver's hand gripping the grip portion before steering, and the discharge gas can be quickly stored in the gas storage recess, so that measurement time can be shortened and the presence or absence of alcohol component discharged from the driver can be detected stably in a short time.

Furthermore, in the intoxication detection device having the configuration described above, it is preferable that a discharge passage capable of discharging the discharge gas exhausted by the exhaust fan to the outside is disposed at a position facing the gas detection sensor with the exhaust fan interposed therebetween, because the air or the discharge gas stored in the gas storage recess can be discharged to the outside efficiently and quickly through the discharge passage.

Furthermore, in the intoxication detection device having the configuration described above, it is preferable that the grip detection sensor that detects the grip or no-grip by the driver is disposed in an area of the grip portion that can be covered with the hand of the driver simultaneously during the measurement of the discharge gas by the gas detection sensor, because it is possible to detect, by the grip detection sensor, the grip state (grip or no-grip, and the like) of the grip portion by the driver simultaneously with the measurement of the discharge gas, so that it is possible to detect the intoxication of the driver more accurately.

### BRIEF DESCRIPTION OF THE DRAWINGS

The present invention will become more fully understood from the detailed description given hereinbelow and the accompanying drawing which is given by way of illustration only, and thus is not limitative of the present invention and wherein:

FIG. 1 is a schematic plan view illustrating a steering wheel with an intoxication detection device mounted therein, according to an embodiment of the present invention;

FIG. 2 is a partially enlarged plan view of an area in the steering wheel in FIG. 1 which includes the intoxication detection device mounted;

FIG. 3 is a schematic cross-sectional view taken along in FIG. 2; and

FIG. 4 is a block diagram of the intoxication detection device according to the embodiment.

### DETAILED DESCRIPTION OF THE INVENTION

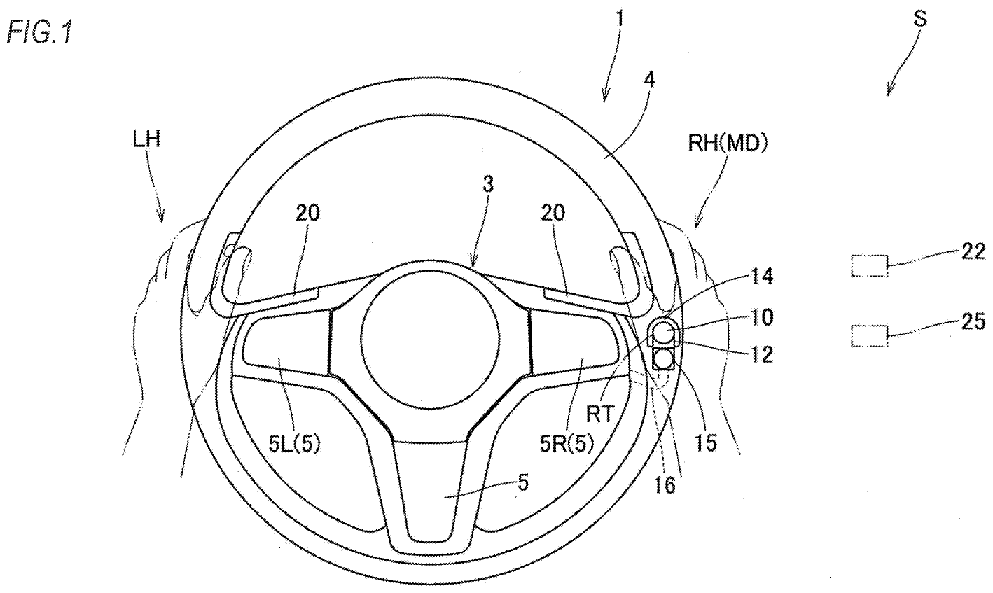

An embodiment of the present invention will be described below with reference to the drawings. As illustrated in FIG. 1, an intoxication detection device S in the embodiment is mounted in a steering wheel 1 steered by a driver MD in a vehicle.

As illustrated in FIG. 1, the steering wheel 1 includes a boss portion 3 connected to a rotation shaft (not illustrated), a ring portion 4 in a substantially annular shape disposed around the boss portion 3 to serve as a grip portion for gripping during steering, and a plurality (three, in this embodiment) of spoke portions 5 connecting the boss portion 3 and the ring portion 4.

The intoxication detection device S includes a gas detection sensor 10, a gas storage recess 12 accommodating the gas detection sensor 10 and storing discharge gas discharged from the skin of the driver MD, a touch sensor 14 serving as a placement check sensor that detects (checks) a state in which a hand is blocking the gas storage recess 12, an exhaust fan 15, a discharge passage 16, a grip detection sensor 20 that detects a grip or no-grip by the driver MD, and a control device 25 that controls the operations of the gas detection sensor 10 and the exhaust fan 15. According to the embodiment, the intoxication detection device S is disposed in an intersecting area (the area in contact with the right hand RH of the driver MD) between the ring portion 4 and a right spoke portion 5R, as illustrated in FIG. 1. Specifically, this area is an area where the thenar RT of the right hand RH comes into contact when the driver MD grips the ring portion 4. Specifically, when the steering wheel 1 is viewed from above, the intoxication detection device S is disposed such that the gas detection sensor 10 is positioned on the right side of the center of the spoke portion 5R in a width direction and the exhaust fan 15 and the discharge passage 16 are positioned in series under the gas detection sensor 10 (see FIGS. 1 and 2).

The gas detection sensor 10 can detect the alcohol (ethanol) component contained in the discharge gas discharged from the palm (skin) of the right hand RH of the driver MD gripping the ring portion 4 without contacting the right hand RH, and a commercially available semiconductor product is used in the embodiment. Specifically, the gas detection sensor 10 has a substantially disk shape with an outer diameter d of about 7 mm (see FIG. 2). In the embodiment, a semiconductor-type sensor is used as the gas detection sensor 10, but a fuel cell type or a non-diffusion infrared absorption type may also be used. Operations are controlled by the control device 25 in the intoxication detection device S of the embodiment such that the gas detection sensor 10 measures the discharge gas stored in the gas storage recess 12 a predetermined period of time (specifically, about 5 seconds) after the exhaust fan 15 stops operating.

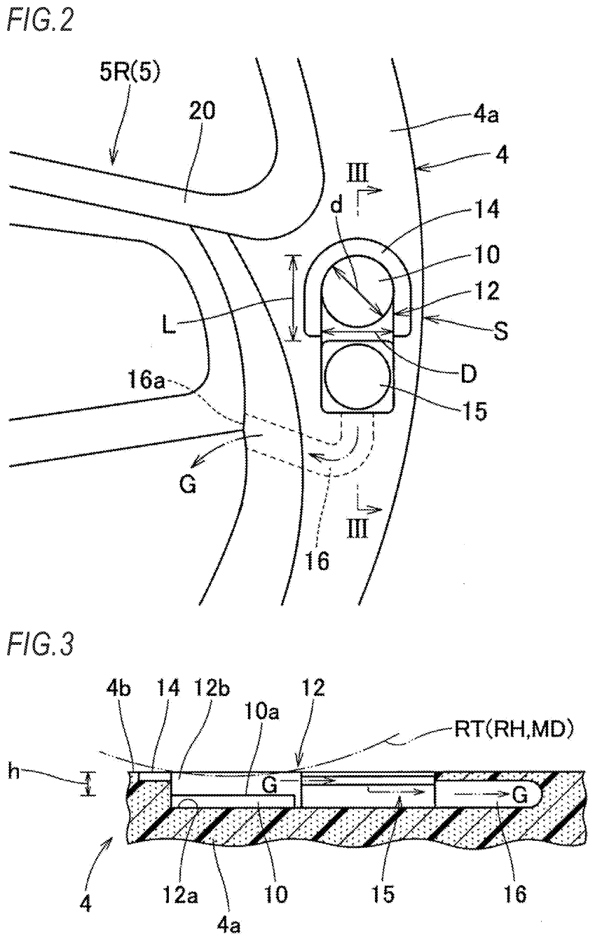

As illustrated in FIG. 3, the gas storage recess 12 is formed to be partially recessed from a surface 4b of the ring portion 4 (specifically, from a coating layer 4a forming the surface 4b side of the ring portion 4). That is, the gas storage recess 12 has an opening 12b on the surface 4b side of the ring portion 4. The gas detection sensor 10 is disposed on a bottom surface 12a side (front end side of the gas storage recess 12), and the depth of the recess is set such that the gas detection sensor 10 disposed on the bottom surface 12a side does not contact the right hand RH (specifically, the thenar RT of the palm) of the driver MD that contacts a peripheral edge 12c of the opening 12b (i.e., that blocks the opening 12b of the gas storage recess 12). Specifically, as illustrated in FIG. 2, the gas storage recess 12 has a substantially groove-like shape extending substantially in the vertical direction in an outer shape of the steering wheel 1 viewed from above during straight-ahead steering, in which the upper end side is formed in a substantially semi-circular shape, and the gas detection sensor 10 is disposed on the bottom surface side of the upper end side. Specifically, the width dimension D of the opening (see FIG. 2) of the gas storage recess 12 is set to be substantially the same as the outer diameter dimension d of the gas detection sensor 10, and the distance h (see FIG. 3) between a surface 10a of the gas detection sensor 10 disposed on the bottom surface 12a side and the surface 4b of the ring portion 4 is set to about 2 mm. Further, the length dimension L (see FIG. 2) of the gas storage recess 12 is set slightly larger than the outer diameter dimension d of the gas detection sensor 10. Then, the discharge gas discharged from the right hand RH of the driver MD fills the inside of the gas storage recess 12 for about 5 seconds after the right hand RH (the thenar RT) comes into contact with the peripheral edge 12c of the opening 12b (i.e., after the contact is detected by the touch sensor 14).

The touch sensor 14 forms the placement check sensor that detects (checks) the state in which the hand is blocking the gas storage recess 12, and in this embodiment, the touch sensor 14 is disposed in the area of the peripheral edge 12c of the opening 12b in the gas storage recess 12 so as to detect the contact state of the right hand RH (thenar RT) with the peripheral edge 12c of the opening 12b. A capacitive sensor, an electrical impedance sensor, or a resistive film sensor can be used as the touch sensor 14, and in this embodiment, the capacitive sensor is used.

The exhaust fan 15 is disposed adjacent to the gas storage recess 12, and in this embodiment, adjacent to a lower end side of the gas storage recess 12 when the steering wheel 1 is viewed from above. The exhaust fan 15 is set to operate while the right hand RH (thenar RT) remains in contact with the peripheral edge 12c of the gas storage recess 12 and configured to, during operation, draw in the air and discharge gas G filled in the gas storage recess 12 and discharge the air and discharge gas G toward the discharge passage 16 facing the gas storage recess 12 with the exhaust fan 15 interposed therebetween. In the embodiment, a commercially available product such as a DC sirocco fan is used as the exhaust fan 15. It is to be noted that the exhaust fan 15 is configured such that the exhaust fan 15 in a non-operating state does not block the space between the gas storage recess 12 and the discharge passage 16, and air is allowed to move between the gas storage recess 12 and the discharge passage 16 through the non-operating exhaust fan 15. However, the actual air movement between the gas storage recess 12 and the discharge passage 16 is minimal, that is, it is not likely that the air and the like stored in the gas storage recess 12 is discharged to the outside while the exhaust fan 15 is not in operation. In the intoxication detection device S of the embodiment, the operations are controlled by the control device 25 such that, while the touch sensor 14 is detecting the contact state of the right hand RH (thenar RT) with the peripheral edge 12c of the gas storage recess 12, the exhaust fan 15 is operated for a predetermined period of time (specifically for about 10 seconds) before and after the alcohol component in the discharge gas is measured by the gas detection sensor 10, respectively.

The discharge passage 16 is in communication with the gas storage recess 12 through the exhaust fan 15 and is disposed at a position facing the gas detection sensor 10 with the exhaust fan 15 interposed therebetween. Specifically, according to the embodiment, the discharge passage 16 extends rearward (downward when the steering wheel 1 is viewed from above) from the exhaust fan 15, bends such that a tip side is directed leftward, and passes in a tubular shape through the coating layer 4a to reach the edge of the coating layer 4a, as illustrated in FIGS. 1 and 2. In other words, although detailed illustration is omitted, the discharge passage 16 is not open on the surface 4b side of the coating layer 4a and is open on the tip side away from the exhaust fan 15, in which the discharge gas G exhausted through the exhaust fan 15 can be discharged to the outside from an opening 16a through a boundary portion in the spoke portion 5R between the coating layer 4a and a switch installation portion (not illustrated) (see the two-dotted line in FIG. 2).

The grip detection sensor 20 is provided to detect a grip or no-grip of the ring portion 4 by the driver MD, and is disposed in an area that can be covered with the right hand RH of the driver MD simultaneously with the gas detection sensor 10 when the ring portion 4 is gripped by the driver MD. Specifically, as illustrated in FIG. 1, in the steering wheel 1 during straight-ahead steering, the grip detection sensors 20 continuously extend from the inner edge sides of intersecting portions (corner portions) between the right and left spoke portions 5L, 5R and the ring portion 4, and along the front edge sides of the spoke portions 5L, 5R and the inner edge sides of the ring portion 4 respectively. As illustrated in FIG. 1, while the driver MD is gripping the ring portion 4, with the thenar RT of the right hand RH covering the gas detection sensor 10 (the thenar RT is brought into contact with the peripheral edge 12c of the opening 12b of the gas storage recess 12), the thumb of the right hand RH is usually placed in the corner portion between the spoke portion 5R and the ring portion 4 to contact the grip detection sensor 20. A commercially available capacitive touch sensor is used for the grip detection sensor 20.

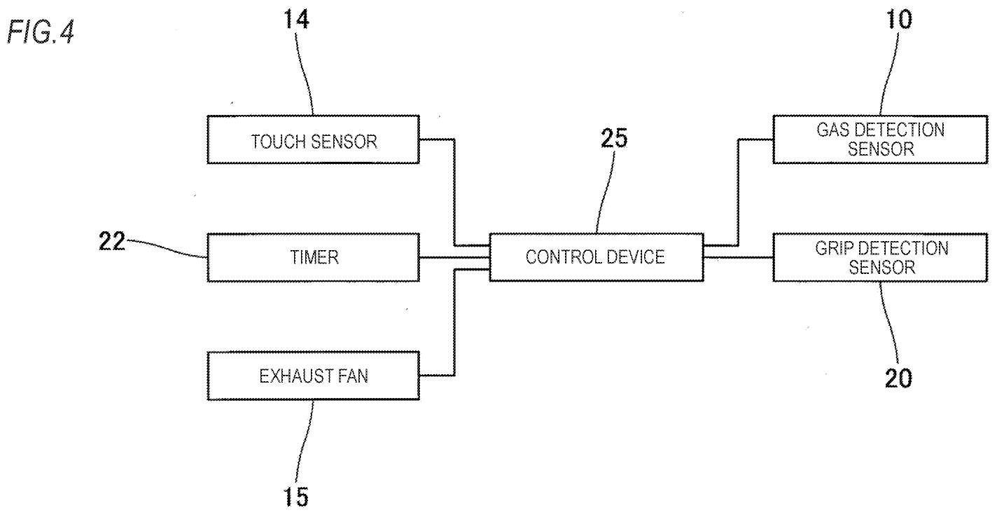

As illustrated in FIG. 4, the control device 25 is electrically connected to the gas detection sensor 10, the touch sensor 14 as a placement check sensor, the exhaust fan 15, and the grip detection sensor 20. Further, the control device 25 is also electrically connected to a timer 22 that measures the lapse of time such as the operating time of the exhaust fan 15 and the time from the end of the operation of the exhaust fan 15 to the start of the measurement by the gas detection sensor 10. According to the embodiment, the gas detection sensor 10 is set so as to measure the alcohol component in the discharge gas three times, and as described above, the exhaust fan 15 is set so as to be operated for the predetermined period of time before and after the measurement by the gas detection sensor 10 while the contact of the right hand RH with the peripheral edge 12c of the gas storage recess 12 is detected. The control device 25 is set so as to determine the intoxication of the driver MD based on the three measurements by the gas detection sensor 10 and the grip or no-grip of the ring portion 4 by the driver MD as detected by the grip detection sensor 20.

According to the embodiment, the intoxication detection device S is configured to store the discharge gas discharged from the right hand RH of the driver MD in the gas storage recess 12 accommodating the gas detection sensor 10 therein, and detect the alcohol component from the discharge gas, in which the intoxication detection device S includes the exhaust fan 15 and is configured to operate the exhaust fan 15 to exhaust the air or the discharge gas stored in the gas storage recess 12 while the opening area of the gas storage recess 12 is blocked with the right hand RH, and then measure, by the gas detection sensor 10, the discharge gas discharged from the right hand RH of the driver MD and stored in the gas storage recess 12. Therefore, it is possible to prevent the detection of alcohol components caused by the factors other than the driver, since it is possible to measure the discharge gas discharged from the right hand RH of the driver MD in such a state that, for example, any air already stored in the gas storage recess 12 before the right hand RH of the driver MD comes into contact, or any discharge gas remaining in the gas storage recess 12 after a predetermined period of time from the previous measurement is exhausted (removed), such that it is possible to accurately detect the alcohol component discharged from the driver MD.

Therefore, according to the embodiment, the intoxication detection device S can prevent misdetection and accurately detect the intoxication of the driver MD.

Further, the intoxication detection device S of the embodiment is configured such that the operations are controlled such that the gas detection sensor 10 measures the alcohol component in the discharge gas a plurality of times (3 times, in this embodiment) within a predetermined period of time. Therefore, compared with the case where the measurement is performed only once, it is possible to prevent misdetection and detect the intoxication of the driver MD more accurately and stably. Of course, if the above is not part of the consideration, the gas detection sensor 10 may be set to perform measurement only once to determine the intoxication.

Furthermore, in the intoxication detection device S of the embodiment, the gas storage recess 12 is disposed in the grip portion (ring portion 4) gripped by the driver MD in the steering wheel 1 steered by the driver MD. Therefore, the gas storage recess 12 can be stably blocked with the hand RH of the driver MD gripping the ring portion 4 before steering, and the discharge gas can be quickly stored in the gas storage recess 12. As a result, the measurement time can be shortened, and the presence or absence of the alcohol component discharged from the driver MD can be stably detected in a short time. In particular, according to the present invention, the intoxication detection device S is disposed in an area of the ring portion 4 where the ring portion 4 intersects the right spoke portion 5R, which is the area that is usually gripped during straight-ahead steering. The thenar RT of the right hand RH comes into contact with this area when the driver MD grips the ring portion 4. That is, according to the embodiment, the gas storage recess 12 is blocked with the thenar RT of the right hand RH coming into contact with the peripheral edge 12c in the intoxication detection device S. The thenar RT has a wide even surface and thus can be stably brought into contact with the surface 4b of the ring portion 4 with the wide surface when the ring portion 4 is gripped, and the contact state with the surface 4b of the ring portion 4 can be easily maintained such that the gas storage recess 12 can be stably blocked for a predetermined period of time. Therefore, the thenar RT is preferably brought into contact with the peripheral edge 12c of the gas storage recess 12. In addition, the intoxication detection device may be disposed near the area in the ring portion where the ring portion intersects the rear spoke portion. Further, the portion of the driver's hand in contact with the peripheral edge of the gas storage recess is not limited to the thenar, and a fingertip or the like may be brought into contact. It is to be noted that the gas storage recess 12 may not be completely blocked with a hand (right hand RH).

Furthermore, in the intoxication detection device S of the embodiment, the discharge passage 16 through which the discharge gas can be discharged to the outside by the exhaust fan 15 is disposed at a position facing the gas detection sensor 10 with the exhaust fan 15 interposed therebetween. Therefore, the air or discharge gas stored in the gas storage recess 12 can be efficiently and quickly discharged to the outside through the discharge passage 16. If the above is not part of the consideration, the discharge passage may be disposed adjacent to the gas detection sensor (gas storage recess) without the exhaust fan interposed therebetween. Further, in the intoxication detection device S of the embodiment, the discharge passage 16 is formed such that the discharge passage 16 is closed on the surface 4b side of the ring portion 4 and extends in a tubular shape through the coating layer 4a, but the outer shape of the discharge passage is not limited to the embodiment, and may be formed in a recess from the surface side of the ring portion in the same manner as the gas storage recess.

Furthermore, in the intoxication detection device S of the embodiment, the grip detection sensor 20 for detecting the grip or no-grip by the driver MD may be disposed in an area of the ring portion 4 that can be covered with the right hand RH of the driver MD simultaneously, during the measurement of the discharge gas by the gas detection sensor 10. Therefore, the grip detection sensor 20 can also detect the grip state (grip or no-grip, or the like) of the ring portion 4 by the driver MD simultaneously with the measurement of the discharge gas so that it is possible to detect the intoxication of the driver more accurately.

In one example of detecting the intoxication of the driver MD by the intoxication detection device S of the embodiment, for example, at the time of engine start before driving, the exhaust fan 15 is operated for a predetermined period of time while the grip state of the ring portion 4 by the driver MD (contact state of the right hand RH with the peripheral edge 12c of the gas storage recess 12) is checked (by detection of the touch sensor 14), and the alcohol component is detected by the gas detection sensor 10 three times with the discharge gas being discharged from the gas storage recess 12 by the operating exhaust fan 15 therebetween, and then the intoxication of the driver MD is determined. In addition, in a configuration in which changes in the driver's state can be detected while the vehicle is traveling with a camera configured to image the driver or a thermocamera configured to measure the driver's body temperature, which may be separately mounted to the vehicle, the camera can be set to operate while the vehicle is traveling to detect the intoxication of the driver.

According to the embodiment, the intoxication detection device S is disposed in the ring portion 4 of the steering wheel 1, but the position of the intoxication detection device is not limited to the ring portion (grip portion) of the steering wheel, and can be disposed, for example, near the shift lever knob or the console box, or near the bezel provided on the spoke portion of the steering wheel. Further, a space for placing a finger or the like can be provided separately around the driver's seat, for example, near a column. Furthermore, in the embodiment, the touch sensor 14 that can detect the contact state of the right hand RH (thenar RT) with the peripheral edge 12c of the opening 12b is used as a placement check sensor for detecting (checking) the state of the hand blocking the gas storage recess 12, but the placement check sensor checking the state of hand is not limited to the touch sensor. For example, when a space for placing a finger or the like is further provided as described above and disposed separately, the placement check sensor is configured with a switch or the like that the driver presses after placing a finger thereon.

Furthermore, in the embodiment, the intoxication detection device is mounted in a steering wheel having an annular ring portion, but this is only an example of the steering wheel, and the intoxication detection device of the present invention can be mounted in a type of steering wheel having a square ring portion or a rod-shaped grip portion partially protruding from the boss portion instead of the ring portion.

Once the driver is determined to be intoxicated, measures such as shutting off the engine may be taken if the vehicle is yet to drive, or measures such as switching to an autonomous driving mode may be taken if the vehicle is traveling and then pulling the vehicle over to stop.

What is claimed is:

1. An intoxication detection device mounted near a driver's seat in a vehicle and configured to detect intoxication of a driver, the intoxication detection device comprising:
a gas detection sensor;
a gas storage recess which is recessed to accommodate the gas detection sensor therein; and
an exhaust fan formed adjacent to the gas storage recess,
wherein the intoxication detection device is configured to detect an alcohol component contained in a discharge gas discharged from a skin of the driver without contacting the skin in response to a hand of the driver being brought close to the gas detection sensor,
wherein the gas storage recess is configured to store the discharge gas therein as the hand covers a peripheral edge in contact therewith,
wherein the exhaust fan is operated for a predetermined period of time while a state of the hand blocking the gas storage recess is detected, and during the operation, the exhaust fan is configured to exhaust air or the discharge gas stored in the gas storage recess, and
wherein the gas detection sensor is set so as to measure the discharge gas stored in the gas storage recess after the exhaust fan stops operating.

2. The intoxication detection device according to claim 1,
wherein operations are controlled such that the gas detection sensor measures an alcohol component in the discharge gas a plurality of times within a predetermined period of time.

3. The intoxication detection device according to claim 1, wherein the gas storage recess is disposed in a grip portion gripped by the driver, in a steering wheel steered by the driver.

4. The intoxication detection device according to claim 3, further comprising
a discharge passage configured to discharge the discharge gas exhausted by the exhaust fan to an outside, the discharge passage being disposed at a position facing the gas detection sensor with the exhaust fan interposed therebetween.

5. The intoxication detection device according to claim 3, further comprising
a grip detection sensor configured to detect a grip or no-grip by the driver,
wherein the grip detection sensor is disposed in an area of the grip portion that can be covered with a hand of the driver simultaneously during the measurement of the discharge gas by the gas detection sensor.
